(19) United States
(12) Patent Application Publication
(10) Pub. No.: US 2024/0379217 A1
(43) Pub. Date: Nov. 14, 2024

(54) SYSTEMS AND METHODS FOR MODEL-BASED DATA ROUTING

(71) Applicant: GE Precision Healthcare LLC, Waukesha, WI (US)

(72) Inventors: Tripti Gupta, Sussex, WI (US); Timothy Lin, Brookfield, WI (US); Sampath Telikicherla Kandala, Marlborough, MA (US); Hongxiang Yi, Excelsior, MN (US)

(21) Appl. No.: 18/661,435

(22) Filed: May 10, 2024

Related U.S. Application Data
(60) Provisional application No. 63/501,618, filed on May 11, 2023.

(51) Int. Cl. G16H 40/20; G16H 20/40; G16H 30/20

(52) U.S. Cl. CPC G16H 40/20; G16H 20/40; G16H 30/20

(57) ABSTRACT

Various methods and systems are provided for adaptably routing radiation therapy data based on a model of a radiation oncology system. In one example, a method comprises defining a model of a radiation oncology system; storing the model in a non-transitory memory of a computing device; sending a query from a source system element to a destination system element to determine a processing capability and an available storage space of the destination system element; selecting a transfer path from the source system element to the destination system element based on the model; transforming data into a format that is compatible with the destination system element; transferring transformed data to the destination system element according to the transfer path; updating the model of the radiation oncology system to reflect a real-time state of transfer; and storing an updated model in the non-transitory memory.

### CROSS REFERENCE TO RELATED APPLICATIONS

The present application claims priority to U.S. Provisional Application No. 63/501,618, entitled “SYSTEMS AND METHODS FOR MODEL-BASED DATA ROUTING”, and filed on May 11, 2023. The entire contents of the above-listed application are hereby incorporated by reference for all purposes.

### FIELD

Embodiments of the subject matter disclosed herein relate to radiation therapy, and more particularly to an adaptable model-based method for routing radiation therapy data.

### BACKGROUND

Healthcare facilities often employ certain types of medical imaging modalities, such as computed tomography, magnetic resonance imaging, ultrasound imaging, and X-ray imaging. These medical imaging modalities user a common format for image data, known as digital imaging and communications in medicine (DICOM®). The evolution of the DICOM® format facilitated the development and expansion of picture archiving and communication systems (PACS). Use of the DICOM® semantics provided by the DICOM® format has become the standard method for managing imaging data access in healthcare institutions.

Radiation oncology may include the administration of targeted radiation to a patient's tumor, for example. Multiple care providers may be involved in the planning and administration of the radiation therapy, with information stored and retrieved from various siloed data sources. A radiation therapy workflow used by care providers may vary among patients based on available resources of a radiation oncology system, such as imaging systems and/or treatment delivery systems which are available for use.

### BRIEF DESCRIPTION

In one example, a method for choosing a data transfer path comprises: defining a model of a radiation oncology system, the model including elements of the radiation oncology system as nodes, and relationships therebetween as edges; storing the model in a non-transitory memory of a computing device; sending a query from a source system element to a destination system element to determine a processing capability and an available storage space of the destination system element; selecting a transfer path from the source system element to the destination system element based on the model of the radiation oncology system and the processing capability of the destination system element; transforming data into a format that is compatible with the destination system element; transferring transformed data to the destination system element according to the transfer path; updating the model of the radiation oncology system to reflect a real-time state of transfer; and storing an updated model in the non-transitory memory.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows a block diagram of an example computing system.

FIG. 2 shows a block diagram of an example radiation oncology system.

FIG. 3 shows a flow chart illustrating a first method for adaptive model-based radiation data routing.

FIG. 4 shows an example model of a radiation oncology system.

FIG. 5 shows a flow chart illustrating a first method for generating a model of a radiation oncology system.

FIG. 6 shows a flow chart illustrating a second method for generating a model of a radiation oncology system.

FIG. 7 shows a flow chart illustrating a first method for adaptive model-based radiation data routing.

FIG. 8 shows a flow chart illustrating a second method for adaptive model-based radiation data routing.

FIG. 9 shows a flow chart illustrating a third method for adaptive model-based radiation data routing.

FIG. 10 shows an example timeline of radiation data routing according to the methods of FIGS. 3-9.

### DETAILED DESCRIPTION

The following description relates to various embodiments of a radiation therapy management system. In particular, systems and methods for adaptable model-based routing of radiation therapy data are provided. A radiation oncology system may include devices, such as imaging systems, treatment planning systems, and treatment delivery systems, from multiple vendors. The devices of the radiation oncology systems may have different capabilities, such as different storage capacities, different data formatting standards, different processing abilities, and so on. During a conventional method for planning and administering radiation therapy, a user (e.g., a healthcare provider) may manually initiate data transfers among devices of the radiation oncology system. For example, when a device produces output data (e.g., a CT imaging device outputs medical image data), a user may manually configure the output data as input data for a subsequent device and manually select the subsequent device. If a device is unavailable, for example if the device is being used to process data from another patient, the user may wait until the device is available, or the user may seek out a different device of the radiation oncology system. This method may create a rigid (e.g., non-flexible) data routing in a workflow, which is time consuming and an inefficient use of resources. Such data routing may be managed manually and without easy implementation of logic or branching.

Described herein are systems and methods for adaptable model-based routing of radiation therapy data in a radiation oncology system. By generating a model of the radiation oncology system, including devices and relationships therebetween, an efficient path for routing patient data through the radiation oncology system may be selected, where the efficient path may have at least one of a lowest processing power demand, a fewest number of steps, and/or a shortest transfer time among transfer paths which route the patient data to desired devices. The model may be updated in real-time as availability of devices of the radiation oncology system change, and a path for routing patient data may be subsequently updated to continue to efficiently route the patient data. In this way, an efficiency and a reliability of patient data transfers within the radiation oncology system may be increased, thus saving time and improving accuracy of data transfers.

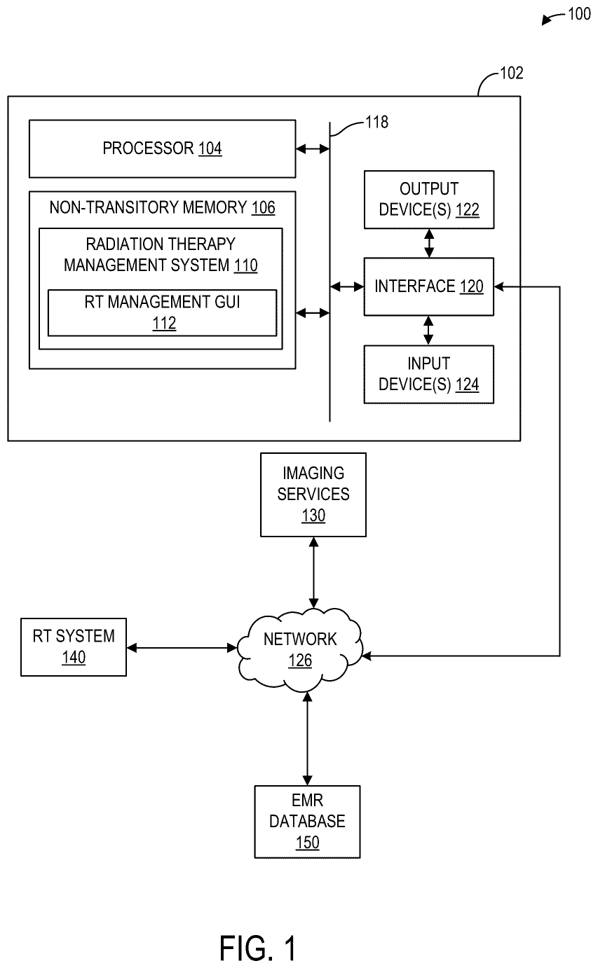

Referring now to FIG. 1, an example of a computing system 100 is shown. Computing system 100 comprises a computing device 102 which may comprise, as illustrative and non-limiting examples, a server, a personal computer, a workstation, a mobile device (e.g., a cellular phone, a smart phone, a computing tablet, and so on), or any other type of computing device.

The computing device 102 includes one or more processors 104 which may be configured to execute machine-readable instructions stored in non-transitory memory 106. Processor(s) 104 may be single core or multi-core, and the programs executed thereon may be configured for parallel or distributed processing. In some embodiments, the processor(s) 104 may optionally include individual hardware components that are distributed throughout two or more devices, which may be remotely located and/or configured for coordinated processing. In some embodiments, one or more aspects of the processor(s) 104 may be virtualized and executed by remotely-accessible networked computing devices configured in a cloud computing configuration. The computing device 102 further includes non-transitory memory 106. It should be appreciated that the computing device 102 may include additional memory devices, including volatile memory, mass storage, local memory, and so on.

Memory 106 includes one or more data storage structures, such as optical memory devices, magnetic memory devices, or solid-state memory devices, for storing programs and routines executed by processor(s) 104 to carry out various functionalities disclosed herein. Memory 106 may include any desired type of volatile and/or non-volatile memory such as, for example, static random access memory (SRAM), dynamic random access memory (DRAM), flash memory, read-only memory (ROM), etc. Processor(s) 104 may be any suitable processor, processing unit, or microprocessor, for example. Processor(s) 104 may be a multi-processor system, and, thus, may include one or more additional processors that are identical or similar to each other and that are communicatively coupled via an interconnection bus. In some examples, memory 106 may include components disposed at two or more devices, which may be remotely located and/or configured for coordinated processing. In some embodiments, one or more aspects of memory 106 may include remotely-accessible networked storage devices configured in a cloud computing configuration. The processor(s) 104 and memory 106 may be coupled, for example, via a communications bus 118.

The computing device 102 may further include an interface 120 communicatively coupled to the processor(s) 104 and memory 106 via the communications bus 118. The interface 120 may be implemented by one or more of any type of interface standard, such as an Ethernet interface, a universal serial bus (USB), a BLUETOOTH interface, a near field communication (NFC) interface, and/or a PCI express interface.

The computing device 102 may further include one or more output device(s) 122 communicatively coupled to the processor 104 and the non-transitory memory 106 via the interface 120. The output device(s) 122 may comprise, for example, one or more display devices. Such a display device may include one or more display devices utilizing virtually any type of technology (e.g., a light emitting diode (LED), an organic light emitting diode (OLED), a liquid crystal display (LCD), a cathode ray tube (CRT) display, an in-place switching (IPS) display, a touchscreen, and so on). In some examples, output device 122 may comprise a computer monitor configured to display medical information of various types and styles. Output device(s) 122 may be combined with processor(s) 104, memory 106, and/or user input device(s) 124 in a shared enclosure, or may be a peripheral display device and may comprise a monitor, touchscreen, projector, or other output device known in the art, which may enable a user to plan and administer radiation therapy according to one or more examples of the current disclosure, and/or interact with various data stored in memory 106.

The computing device 102 may further include one or more user input device(s) 124 coupled to the processor(s) 104 and memory 106 via the interface 120. A user input device 124 may comprise, for example, one or more of a touchscreen, a keyboard, a mouse, a trackpad, a motion sensing camera, a microphone, or other device configured to enable a user to interact with and manipulate data within computing device 102.

The interface 120 may further include a communication device such as a transmitter, a receiver, a transceiver, a modem, a residential gateway, a wireless access point, and/or a network interface to facilitate exchange of data with external machines (e.g., computing devices of any kind) via a network 126. For example, the communication may be via, for example, an Ethernet connection, a digital subscriber line (DSL) connection, a telephone line connection, a coaxial cable system, a satellite system, a line-of-site wireless system, a cellular telephone system, and so on. As a non-limiting example, FIG. 1 shows a plurality of devices/systems that may be communicatively coupled to computing device 102 via network 126, including imaging services 130, a radiation therapy (RT) system 140, and an EMR database 150.

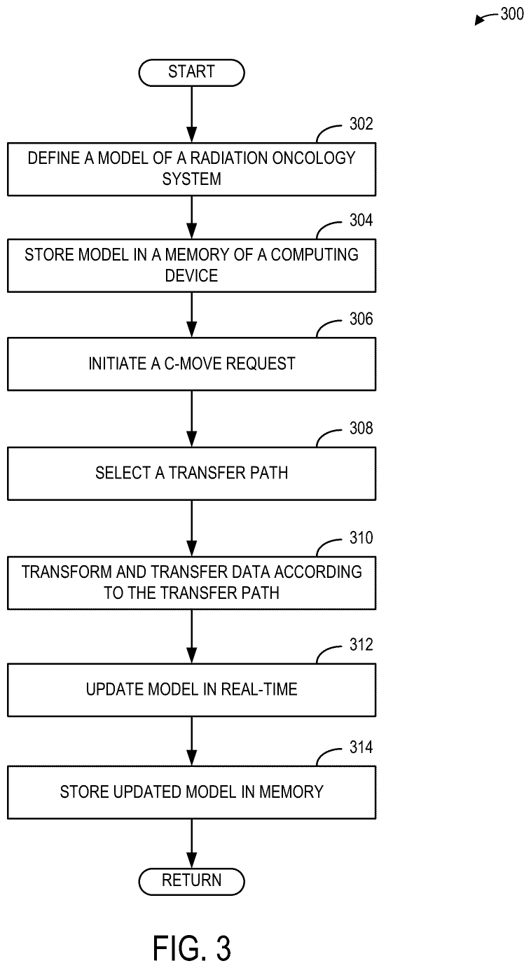

Computing device 102 may further include a radiation therapy management system 110 saved in memory 106. The radiation therapy management system 110 may be configured to define a model of a radiation oncology system in which the computing device 102 is deployed and which may further include at least one of the imaging services 130, the RT system 140, and the EMR database 150. The radiation therapy management system 110 may further be configured to identify an efficient data transfer path among elements of the radiation oncology system, based on the model, and initiate transformation and transferring of data according to the identified data transfer path. Additional information about modeling the radiation oncology system and adaptably routing data based on the model is presented below with respect to FIGS. 3-5.

The imaging services 130 may include a radiology information system (RIS), a picture archive and communication system (PACS), and/or other computing devices associated with diagnostic imaging, including computing devices associated with and configured to control diagnostic imaging scanners. The computing devices comprising the imaging services 130 may manage scheduling of diagnostic imaging exams, recommend and execute scanning protocols to perform diagnostic imaging exams, process imaging data to generate images and/or volumes, save diagnostic images/volumes in memory, process diagnostic images/volumes to segment anatomical regions of interest, etc. Specifically, the RIS may be configured to track and manage radiology requests and workflow (e.g., CT/x-ray imaging requests and workflows) and the PACS may be configured to store diagnostic images obtained during diagnostic imaging sessions (e.g., images obtained during a simulation scan). The imaging services 130 may receive information from the radiation therapy management system 110, such as a filled simulation scan order form dictating a scan protocol to be used to perform a simulation scan (which may be sent to a CT system, for example) and instructions to manage/move diagnostic images (e.g., from a CT system to the PACS and/or to other devices for further processing of the diagnostic images).

The RT system 140 may include an oncology information system (OIS) and/or other devices associated with radiation therapy, such as computing devices associated with and configured to control radiation therapy devices (such as linear accelerators). The OIS may be configured to generate and store oncology-based electronic medical records for patients, including cancer screening, diagnoses, treatments, treatment workflows, image reviews, and so forth. The RT system 140 may receive information from the radiation therapy management system 110, such as a filled radiation therapy plan form dictating parameters of a radiation therapy session (which may be sent to a radiation therapy device, such as a computing device controlling a linear accelerator). Additionally, information from the RT system 140 may be retrieved with the radiation therapy management system 110 and used to autofill the simulation order form and radiation therapy plan form.

EMR database 150 may store EMRs for a plurality of patients. An EMR for a patient may include patient demographic information, family medical history, past medical history, lifestyle information, comorbidities (e.g., preexisting medical conditions), current medications, allergies, surgical history, past medical screenings and procedures, past hospitalizations and visits, etc. EMR database 150 may be an external database accessible via a secured hospital interface, or EMR database 150 may be a local database (e.g., housed on a device of the hospital). EMR database 150 may be a database stored in a mass storage device configured to communicate with secure channels (e.g., HTTPS and TLS), and store data in encrypted form. Further, the EMR database is configured to control access to patient electronic medical records such that editing and access permissions to the electronic medical records is controlled to allow authorized healthcare providers. Additionally, information from the EMR database 150 may be retrieved with the radiation therapy management system 110 and used to autofill the simulation order form and radiation therapy plan form.

While not shown in FIG. 1, one or more workstations may also be communicatively coupled to computing device 102 via network 126. Each workstation may include a processor, memory, communication module, user input device, display (e.g., screen or monitor), and/or other subsystems (similar to the processor, memory, communication module, user input device, and output device of computing device 102) and may be in the form of a desktop computing device, a laptop computing device, a tablet, a smart phone, or other device. Each workstation may be adapted to send and receive encrypted data and display medical information, including medical images in a suitable format such as digital imaging and communications in medicine (DICOM) or other standards. As will be explained in more detail below, the workstations may display graphical user interfaces described herein and may facilitate user interaction with the graphical user interfaces.

The radiation therapy management system 110 may fetch data stored in various data sources, aggregate the data, and share the data with various downstream devices via at least one of multiple connectivity methods. The multiple connectivity methods may include DICOM images, DICOM SRs, HL7, FHIR, proprietary logs associated with a manufacturer or operating system, and so on. Thus, the computing device 102 may be operably coupled to the data sources/downstream devices (e.g., imaging services 130, RT system 140, EMR database 150) via wired connections, wireless connections, and/or any method for communicably connecting systems. As described herein “operably coupled” is to be understood as coupling of elements via connectivity methods (e.g., wired connection, wireless connection, and so on) which enable transfer of data, signals, requests, and/or other information among the operably coupled elements.

It should be understood that computing system 100 shown in FIG. 1 is illustrative and non-limiting, and that another appropriate computing system 100 may include more, fewer, or different components. The various systems included in computing system 100 (e.g., the computing device 102, imaging services 130, RT system 140, and EMR database 150) may be separate devices in communication, as shown in FIG. 1. One or more of the devices described herein may be implemented over a cloud or other computer network. For example, computing device 102 is shown in FIG. 1 as constituting a single entity, but it is to be understood that computing device 102 may be distributed across multiple devices, such as across multiple servers. Further, while radiation therapy management system 110 is shown as being included as part of computing device 102, in some examples, radiation therapy management system 110 may be included as part of one or more other devices, such as implemented as part of imaging services 130 (e.g., in the RIS).

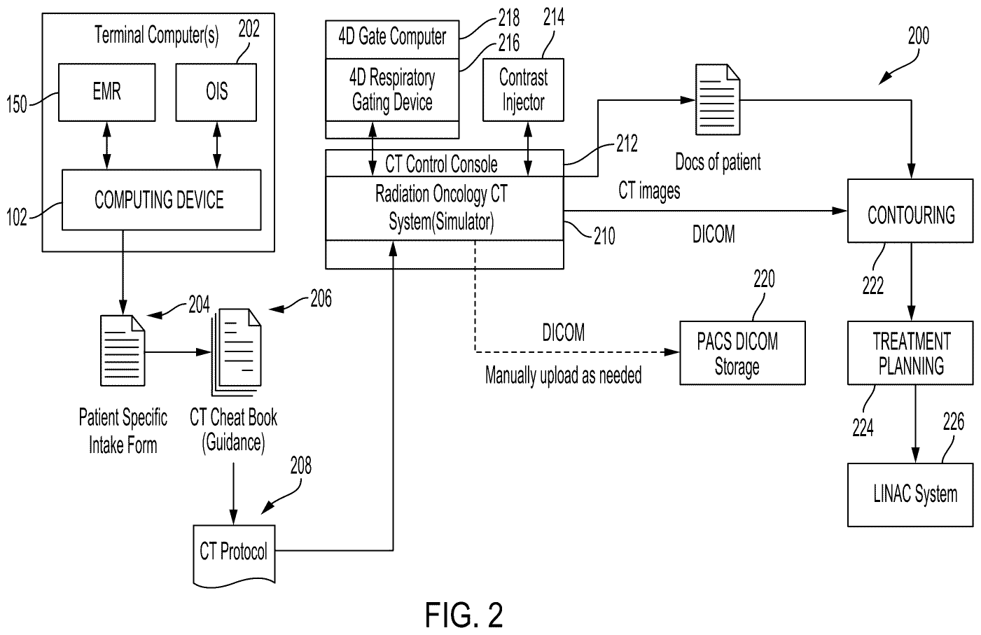

FIG. 2 shows a radiation oncology system 200 that includes aspects of computing system 100. Via the radiation oncology system 200, a patient may be treated with radiation therapy to mitigate cancer, for example. When a patient is treated via the radiation oncology system 200, a workflow may be followed that starts with patient registration. During the registration procedure, a user (e.g., office personnel) may enter patient information to the computing device 102 or another workstation, which sends the patient information to the EMR database 150 and/or an OIS 202 (which as explained above may be implemented on a device that is part of the RT system 140). The patient information may include patient identifying information (e.g., name, date of birth) as well as patient demographic data (e.g., age, gender). The patient information may be gathered with and/or aggregated into a patient-specific intake form 204.

A simulation procedure may be performed on the patient and is planned through the radiation therapy management system 110. For planning the simulation procedure, the RT management GUI 112 may include a simulation order form, which may be part of or separate from the patient-specific intake form 204. A user (e.g., the radiation oncologist) may create the plan for the simulation via the simulation order form. A CT scan may be performed during the simulation to produce a 3D model of the patient's anatomy for guiding the radiation therapy. As shown in FIG. 2, the information from the patient-specific intake form 204 and/or simulation order form may be used along with CT protocol guidance 206 to generate a CT protocol 208, and a CT system 210 may be controlled (by a CT control console 212) according to the CT protocol 208 to perform the CT scan. The CT control console 212 may include a display device configured to display an imaging interface. In some examples, the CT protocol 208 may be displayed via the imaging interface and/or additional information of the simulation scan, as dictated by the information obtained via the RT management GUI, may be displayed via the imaging interface. The imaging interface may be specific to the CT system and thus may not display radiation therapy planning information. As such, the imaging interface may have increased display area relative to the RT management GUI available for CT-specific information not displayed via the RT management GUI. In this way, the RT management GUI may display a limited amount of CT-related information (e.g., the simulation order form/summary of the simulation order form) that is relevant to radiation therapy planning.

In addition to the CT system 210, the CT scan may be facilitated by a contrast injector 214 (which may inject a contrast agent into the patient prior to the CT scan commencing). Further, a respiratory gating device 216 (controlled by a gate computer 218) may be included to enable respiratory gating of the radiation therapy. The images obtained during the simulation may be saved in a suitable image archive (e.g., a PACS 220) and sent for further processing to generate the 3D model of the patient's anatomy. The further processing may include contouring performed by a contouring module 222, as shown in FIG. 2. Once the 3D model is available, radiation therapy treatment may be planned using a treatment planning module 224.

Using the radiation therapy management system 110, a radiation oncologist may create a treatment plan for treating the patient that specifies the area of interest for radiation treatment as well as the radiation modality to be used. Specifically, the radiation therapy management system 110 may generate and/or display the RT management GUI 112, which may include a plan intent form. The radiation oncologist may create the treatment plan via the plan intent form of the RT management GUI 112.

For delivery of the radiation therapy, the radiation therapist administers the radiation therapy using a linear accelerator (LINAC) system 226, for example. The RT management GUI 112 may be viewed during administration of the radiation therapy, highlighting the treatment area on a 3D human figure displayed on the GUI. After treatment delivery, regular check-ups are used to keep track of the patient's progress, and the RT planning GUI may be used to plan any follow-up care.

In the example shown in FIG. 2, the computing device 102, CT control console 212, and gate computer 218 may be located in the same physical location (e.g., within a CT control room, which may be adjacent a room housing the CT system). The PACS 220, LINAC system 226, and computing device(s) implementing the contouring module 222 and treatment planning module 224 may be located in a different physical location(s) from the CT control room. Further, it is to be appreciated that the EMR database 150 and OIS 202 may be located at a different physical location(s) than the computing device 102 and that each of the EMR database and the OIS 202 may be implemented with a cloud-based platform and accessed via the computing device 102 or another workstation.

Thus, the RT management GUI shows all pertinent data from the OIS and EMR systems during this workflow, including schedule data, simulation order data, demographic data, service data, and data on modality and services. The RT management GUI may present the various pertinent data in a common, human-readable format. The radiation therapy management system may be configured to retrieve the pertinent data from the different data sources (e.g., the OIS and EMR database) in one or more first formats and convert the data to the common, human-readable format. When the simulation order form and plan intent form are filled out, the radiation therapy management system may be configured to convert the simulation order form (or the information obtained via the simulation order form) and/or the plan intent form (or the information obtained via the plan intent form) into one or more second formats (different than the common, human readable format and in some examples, different than the one or more first formats). For example, the simulation order form/information from the simulation order form may be converted to a second format usable by the CT control console/CT system.

A patient treatment workflow may include a series of steps that are selected by one or more healthcare provider, and/or automatically selected using a treatment planning algorithm, to monitor a patient, provide treatment, and/or plan further treatment for the patient. The patient treatment workflow may be provided to one or more devices of a radiation oncology system and may be implemented as executable instructions to execute the series of steps of the workflow. As described herein, the radiation oncology system may include multiple devices that are configured to perform different healthcare related tasks, such as imaging, treatment planning, and treatment delivery. The patient treatment workflow may define an ordered series of steps to be implemented for a given patient case. Devices used to execute the different steps of the patient treatment workflow may be included in different departments of the radiation oncology system, and may therefore be in direct and/or indirect communication with each other. For example, the patient treatment workflow may include receiving patient information at a user interface of a first department, performing an imaging scan using a CT system of a second department, providing treatment to the patient using a treatment delivery device of a third department, segmenting captured images of the patient using a segmentation device of a fourth department, and so on. Each department may include one or more different users (e.g., radiology technicians, oncologists, and so on) who have different qualifications to perform different tasks in the radiation oncology system and in the patient treatment workflow. Conventional methods for transferring data among devices in different departments of the radiation oncology system include a user manually inputting a command into a first device of a first department to send data to a second device of a second department, according to the ordered series of steps of the patient treatment workflow.

Methods described herein for adaptable routing of radiation therapy data include automatically transmitting data for a patient according to a patient treatment workflow using a model of a radiation oncology system. A computing device of the radiation oncology system may receive information of a patient treatment workflow, including an ordered series of steps for patient treatment. The method may generate a model of the radiation oncology system, including devices of each department of the radiation oncology system and information about a processing capability, available storage space, and a data format for each device. The method may identify and automatically direct data transmission among devices of the radiation oncology system, according to the patient treatment workflow, using an automatically selected transfer path from the model. For example, a first step of the patient treatment workflow may include receiving patient information at a computing device (e.g., via a patient intake form). The patient treatment workflow may include, at a second step, performing a CT scan. The method described herein may automatically identify a CT system of the radiation oncology system, using the model thereof, and send the patient information from the computing device to the CT system. The patient treatment workflow may further include storing CT images in a PACS of the radiation oncology system. Following completion of the CT scan, the method may automatically identify the PACS (e.g., using the model of the radiation oncology system), and send the CT images from the CT system to the PACS. In this way, data used in and captured using steps of the patient treatment workflow may be automatically sent among devices and departments of the radiation oncology system without user input to direct data transmission. As further described herein with respect to FIGS. 3-10, one or more devices of one or more departments of the radiation oncology system may be unavailable for data processing and/or data storage as requested by the patient treatment workflow. The adaptive nature of the method described herein and the model of the radiation oncology system enables automatic identification and selection of a data transfer path that includes devices with processing capabilities and data storage availability as requested by the patient treatment workflow. This may increase a speed of data transmission, reduce delays and/or repetitions in data transmission, and increase data transfer efficiency. Data transfers to one or more devices may be synchronous and/or asynchronous. Further, one or more data may be transferred simultaneously.

In another example, the method for adaptable routing of radiation therapy data may identify and automatically direct data transmission among applications and/or modules of one or more devices of the radiation oncology system, according to the patient treatment workflow, using an automatically selected transfer path from the model. For example, a scanning device may include a first processing application, a second processing application, and a third processing application. Each of the processing applications may include one or more different steps of a method for processing an image captured by an image scan. The method for adaptable routing of radiation therapy data may include automatically identifying which of the first, second, and third processing application is configured to most efficiently process the image (e.g., fastest, least processing power demand, most available storage) and automatically route image data to the identified and selected application.

In conventional radiation therapy data routing methods, a user may select a destination (e.g., a scanning device, a treatment device, an imaging device, a data storage device) to send image data to (e.g., from a scanner, from PACS). The user may manually initiate this data transmission. However, the user may select a destination that is not configured to receive and/or process the data. For example, the data may be in a different format than the destination is configured to receive and/or process. In another example, the destination device may not have a processing and/or a storage capacity to receive and/or process the data. The methods described herein provide automated surveying of device storage and processing capacity. The methods further provide automatic identification and selection of devices, and automatic sending of data to selected devices. In this way, the method removes user error that may result in inefficient data processing. For example, where a user may select and send data to a device that does not have a demanded processing and/or storage capacity, the methods for automatic model-based data routing identify and send data to devices that do have desired processing and/or storage capacity. The user selection-based methods may result in an increase in bandwidth and/or data processing demand, as well as an increase in network traffic, as a result of sending the data to a device that does not have demanded capacity. The methods described herein address this challenge and avoid this increase in bandwidth, processing demand, and network traffic by sending to automatically selected modules and/or devices.

The methods described herein may use DICOM® as the standard for medical imaging, including the DICOM® command set, data formats, interface specifications, communication protocols, and command syntax. DICOM® sets forth information objects (types of data, such as CT, magnetic resonance, x-ray, ultrasound, etc.), service classes (actions with data, such as send, receive, print, etc.), and data transmission protocols. DICOM application services provide the ability to transfer images and image related data between DICOM® applications. A DICOM® service-object pair (SOP) is used to push and/or pull information between DICOM® applications.

One example device that may include a DICOM® application is a PACS. PACS have a number of roles in data management, including receiving image data sets fed from imaging devices and managing storage systems for data persistency, both short term and long term storage. PACS accept query requests from client applications, enabling those client applications to retrieve specified data. PACS may also interface with other healthcare information systems. Various devices may communicate with a PACS via the DICOM® standard, including medical imaging devices, workstations, RIS devices, and so on. A device that generates, communicates, transfers, and/or stores data according to the DICOM® standard may be referred to herein as a DICOM® device, and may include PACS, imaging devices, RIS devices, workstations, and any other device that includes a DICOM® application or is otherwise configured to handle data according to the DICOM® standard.

Turning now to FIG. 3, a flowchart illustrating a method 300 for adaptably routing radiation therapy data based on a model of a radiation oncology system is shown. Method 300 may be executed according to instructions stored in memory of a radiation therapy management system of a computing device and executed by one or more processors of the computing device, such as computing device 102 of FIG. 1.

At 302, method 300 includes defining a model of a radiation oncology system. As briefly described with respect to FIGS. 1 and 2, the computing device which stores radiation therapy management system may be included in a radiation oncology system. The radiation oncology system may further include at least one imaging system (e.g., the CT system 210 of FIG. 2), at least one treatment planning system (e.g., the treatment planning module 224), and at least one treatment delivery system (e.g., the LINAC system 226). One or more of the imaging system, the treatment planning system, and the treatment delivery system may use different data formats and protocols, which may pose challenges when transferring data therebetween. Additionally, in radiation oncology systems which include more than one imaging system, for example, a first imaging system may use a first data format and have a first processing and/or storage capability, while a second imaging system may use a second data format and have a second processing and/or storage capability which is different from the first imaging system. Similarly, systems having more than one treatment delivery system and/or more than one treatment planning system may have different data formats and/or processing and storage capabilities for each system, further complicating data transmission therebetween. Defining the model of the radiation oncology system includes generating a graphical model of the radiation oncology system, where each element (e.g., each imaging system, treatment delivery system, and treatment planning system) is defined as an individual node. Relationships among elements are defined as edges among the nodes. The model further includes defined capabilities of each element (e.g., data transfer, processing, and/or storage capabilities).

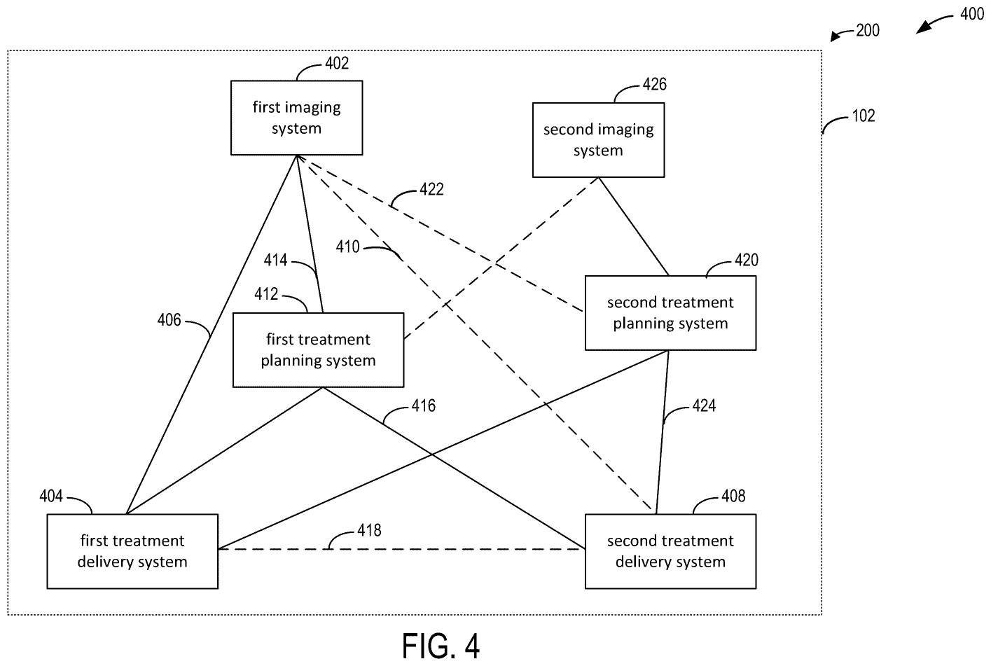

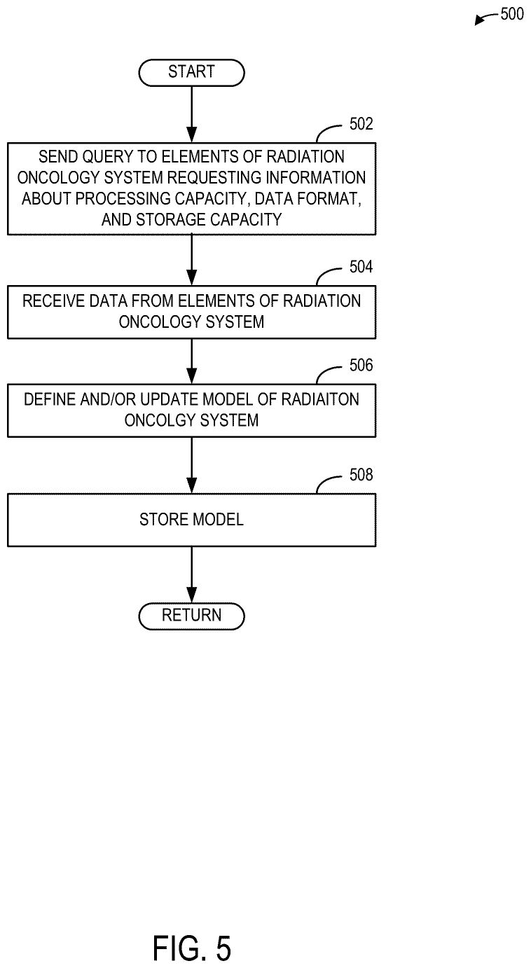

Turning briefly to FIG. 4, an example model 400 of the radiation oncology system 200 is illustrated. The computing device 102 of FIGS. 1-2 is communicably coupled to each of the elements of radiation oncology system, as illustrated by a dotted line box that includes each of the elements. In this way, instructions stored in memory and executed by one or more processors of the computing device 102 may be used to control and direct data transmission among elements of the radiation oncology system as described with respect to FIG. 3 and further described with respect to FIGS. 5-9. Each imaging system, treatment delivery system, and treatment planning system are defined as an individual node, and relationships therebetween are described as edges. Relationships among elements may be defined by capabilities of respective elements. For example, a first imaging system 402 is communicably connected to a first treatment delivery system 404 by a first edge 406, and is communicably connected to a second treatment delivery system 408 by a second edge 410. The first edge 406 is a solid line, which may indicate that a data format used by the first imaging system 402 is the same as a data format used by the first treatment delivery system 404. The second edge 410 is a dashed line, which may indicate that a data format used by the first imaging system 402 is different from a data format used by the second treatment delivery system 408. The first imaging system 402 is further communicably coupled to a first treatment planning system 412 by a third edge 414. A second imaging system 426 is communicably coupled to the first treatment planning system 412 and a second treatment planning system 420. The second treatment delivery system 408 is communicably coupled to the first treatment planning system 412 by a fourth edge 416 and is communicably coupled to the first treatment delivery system 404 by a fifth edge 418. In this way, the model 400 of the radiation oncology system illustrates relationships among elements, as well as capabilities of each element, which are further described with respect to FIGS. 3 and 5.

Returning to FIG. 3, at 304, the method 300 includes storing the model in a memory of the computing device. For example, the model 400 shown in FIG. 4 may be generated at operation 302 and stored in the non-transitory memory 106 of the computing device 102 at operation 304.

At 306, the method 300 includes initiating a C-MOVE request, which may include inducing a source workflow step to send a query from a source system element to a destination system element to determine a processing capability and an available storage space of the destination system element. C-MOVE is a DICOM® operation that allows an application entity to instruct another application entity to transfer stored SOP instances to another application entity using a C-STORE operation. The source system element may be an imaging system and the destination system element may be a treatment planning system. The source workflow step may indicate a desired source system element which is a data source, and a desired destination system element or type of destination system element. For example, the source workflow step (e.g., as indicated by the patient treatment workflow) may indicate that CT data is to be sourced from a first imaging system and delivered to a treatment planning system configured to perform a treatment planning step. The radiation oncology system may include multiple treatment planning systems that are configured to perform the treatment planning step requested by the patient treatment workflow. The C-MOVE request thus queries elements of the radiation oncology system to identify one or more elements that are configured to perform a step requested by the patient treatment workflow and have a desired processing capability and storage capability. The model generated at operation 302 may include predetermined paths among elements of the radiation oncology system. For example, referring to FIG. 4, the first imaging system 402 may be configured to transfer data to the first treatment planning system 412, and the first treatment planning system 412 may be configured to transfer data to the first treatment delivery system 404. As an alternate path, the first imaging system 402 may be configured to transfer data to the second treatment planning system 420, and the second treatment planning system 420 may be configured to transfer data to the second treatment delivery system 408. Using predetermined paths indicated by the model and information received from querying the elements, the method 300 may identify and select a data transfer path that efficiently performs steps of the patient treatment workflow (e.g., least number of data transmission steps, least number of data format changes, fastest processing time, etc.).

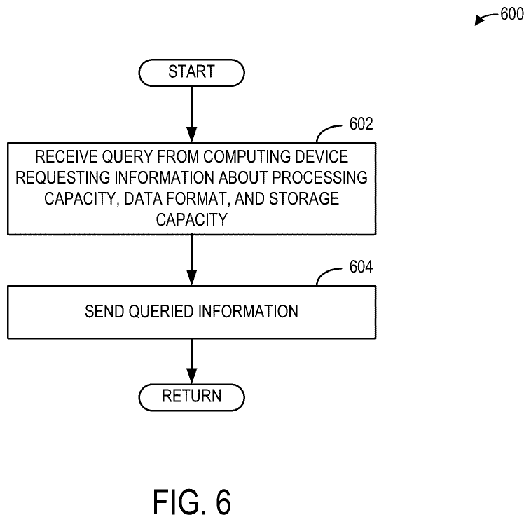

As further described with respect to FIGS. 5-6, the computing device 102 may send a query to one or more elements of the radiation oncology system that requests information about a processing capacity, data format, and storage capacity of each respective element. The queried elements may return requested data, which may be used to define a model of the radiation oncology system. For example, with reference to FIG. 4, the computing device 102 may send a query to the first imaging system 402 and to the first treatment planning system 412 to determine a processing capability and an available storage space thereof. The first treatment planning system 412 may indicate that it has the processing capability and available storage space to receive and process data from the first imaging system 402. In some examples the computing device 102 may further send queries to all destination system elements of a desired type at 306. For example, the computing device 102 may send a query to each of the first treatment planning system 412 and the second treatment planning system 420 to determine a processing capability and an available storage space of each treatment planning system. A sixth edge 422 between the first imaging system 402 and the second treatment planning system 420 is dashed, which may indicate that the first imaging system 402 and the second treatment planning system 420 use different data formats, for example. The dashed line of the sixth edge 422 may indicate an additional or alternate mismatch in the relationship between the elements which may cause challenges in data transfer therebetween.

At 308, the method 300 includes selecting a transfer path. The transfer path may be selected from paths identified in the radiation oncology system from the source system element to a destination system element using the model generated at operation 302. The selected transfer path may be selected based on an efficiency of the path, including a processing capability and an available storage of the destination system element as shown in the model and determined using the C-MOVE request queries. For example, selecting the transfer path includes comparing a processing power demand, number of steps, and transfer time among a two or more paths from the source system element to the destination system element as defined by the model, and selecting the transfer path at least one of a lowest processing power demand, a fewest number of steps, and/or a shortest transfer time.

In the example described above with respect to FIG. 4, a first path from the first imaging system 402 to the first treatment planning system 412, and from the first treatment planning system 412 to the first treatment delivery system 404 may have the fewest number of steps, as the data format used by each of the elements is the same and therefore data may not be transformed prior to or during transfer. However, one or more of the elements of the first path may have a reduced capability. For example, the first treatment planning system 412 may be in use to process one or more data sets prior to being available to process data from the first imaging system 402. Additionally or alternatively, a storage capacity of an element (e.g., the first treatment delivery system 404) may be full and thus unable to receive data from the first treatment planning system 412. Processing and/or storage of data from the first imaging system 402 according to the first path may thus be delayed until element capability is restored. The method 300 may recognize (e.g., during operations 306 and 308) that the first path may not be a most efficient data transfer path and may select an alternative path provided by the model. For example, if it is determined that the first treatment planning system 412 is not capable of processing data from the first imaging system 402, a second path may be selected, where the second path directs data from the first imaging system 402 to the second treatment planning system 420. The second path may further include directing data from the second treatment planning system 420 to the second treatment delivery system 408 via a seventh edge 424.

Following selection of the transfer path, at 310, the method 300 includes transforming and transferring data according to the transfer path. Data, such as medical images and related data from the imaging system, may be transformed into a format which is compatible with a destination element format. For example, the imaging system may use a DICOM® standard while a treatment planning system uses a HL7 standard. When data is formatted to be compatible with the destination element, data is transferred according to the selected transfer path. Data may be transferred using a variety of protocols, such as UPS-RS, DICOM®-UPS, and a modality worklist adaptor.

At 312, the method 300 includes updating the model (e.g., generated at operation 302) in real time. For example, as data transfer progresses according to the selected transfer path, the model is updated to reflect a current state of transfer. An efficiency of the transfer path (e.g., the first transfer path) may be compared to an updated model of the radiation oncology system to select a second transfer path from the source system element to the destination system element based on the updated model when the second transfer path is more efficient than the transfer path. The second path may be automatically chosen due to an availability of the second treatment planning system when the first treatment planning system is unavailable. However, during processing of data by the first treatment planning system, the second treatment delivery system (e.g., indicated in the second path) may become unavailable, for example, is being used to process other data and/or a storage capacity is full. The model may be updated to indicate that the second path may no longer be a most efficient data transfer path. Paths indicated by the model may be reevaluated, as described with respect to operations 306 and 308 to identify and select a transfer path where the element at which the path was halted (e.g., the first treatment planning system) is the source workflow step (e.g., the source system element) and the destination system element may change, depending on a processing capability of the destination system element. In this way, an efficiency and reliability of data transfer may be maintained and/or increased during real-time changes in the radiation oncology system.

At 314, the method 300 includes storing the updated model in the memory of the computing device. For example, the model generated at operation 302 and stored in the non-transitory memory 106 of the computing device 102 at operation 304 may be replaced by the updated model generated at operation 312. Alternatively, the model generated at operation 302 may be adjusted to reflect the updated model from operation 312.

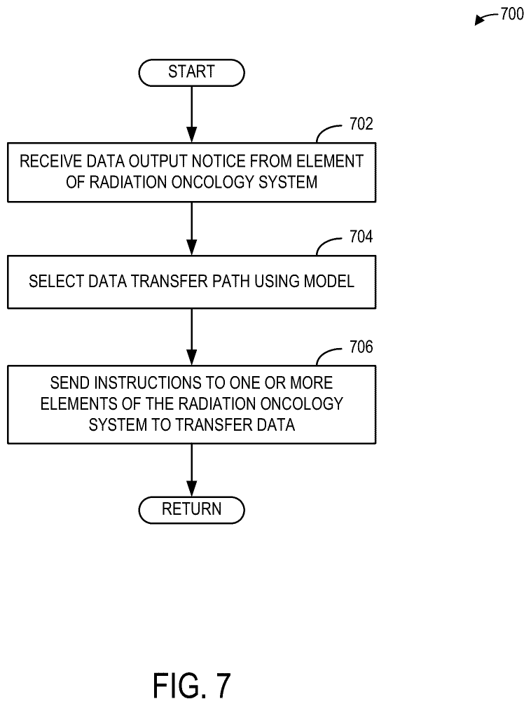

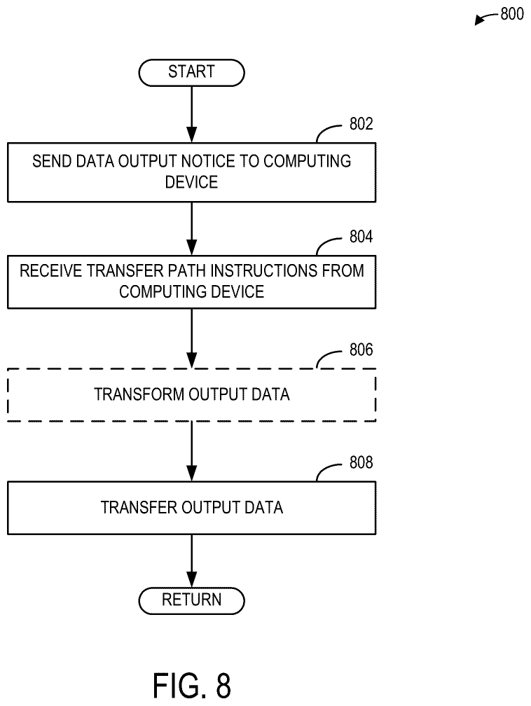

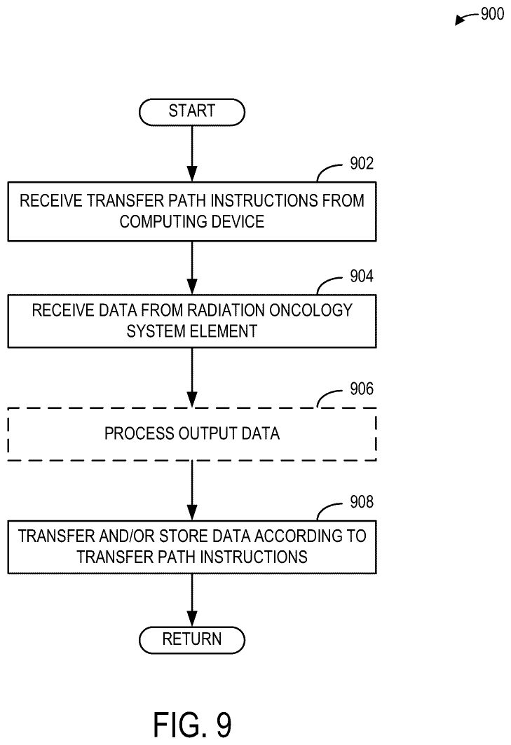

An example process for adaptable routing of radiation therapy data using a model of a radiation oncology system is described herein with respect to FIGS. 5-9. The model may be generated by a computing device of a radiation oncology system, such as the computing device 102 described with respect to FIGS. 1-4. The model may be generated as described with respect to the method 300 of FIG. 3, and as further described with respect to a method 500 of FIG. 5. The model is further generated using data from one or more elements of the radiation oncology system, herein also referred to as queried elements. The queried elements may execute a method 600 of FIG. 6 to send information about a processing capacity, a data format, and an available storage space of a respective queried element to the computing device, where the information is used to generate and/or update the model of the radiation oncology system. The model may be continuously updated in response to receiving updated information about each queried element. Following generation of the model, the computing device may receive a notice from a queried element that a first element of the radiation oncology system has a data output. In response, the computing device may consult the model of the radiation oncology system, select a data transfer path, and send instructions to one or more elements of the radiation oncology system to facilitate data transfer throughout elements of the radiation oncology system, as described with respect to a method 700 of FIG. 7. The first element may execute a method 800 of FIG. 8 to notify the computing device of output data, receive transfer path instructions from the computing device, and output the output data according to the transfer path instructions. The output data may be output to a second element of the radiation oncology system. The second element may execute a method 900 of FIG. 9 to receive the output data from an element of the radiation oncology system (e.g., the first element), process the data, and transfer and/or store the output data, according to the instructions from the computing device. The method 900 may be executed by more than one elements of the radiation oncology system to receive, process, and/or store received data.

As briefly described above, the model of the radiation oncology system, including different transfer paths that are available at a given time, may be updated in response to changes in processing capability and/or available storage space from one or more queried elements. Thus, the methods 500 and 600 may be executed continuously while the methods 700, 800, and 900 are executed. In this way, instructions for data transfer sent by the computing device to elements of the radiation oncology system may indicate data transfer paths that are fast and efficient. For example, data transfer paths may be selected that include a least number of data format transformations, a least number of involved elements of the radiation oncology system, and/or a shortest duration of processing time.

FIG. 5 shows a flow chart for the method 500 for generating a model of a radiation oncology system. The model may be an example of a model generated according to the method 300, such as the model 400. The method 500 may be executed according to instructions stored in memory of, and executed by one or more processors of a computing device of the radiation oncology system. For example, the method 500 may be executed by the computing device 102.

At 502, the method 500 includes sending a query to elements of the radiation oncology system requesting information about a processing capacity, a data format, and a storage capacity of a queried element. The query may be sent via a wired and/or a wireless connection between the queried element and the computing device. In some examples, the query may be simultaneously sent to each element of the radiation oncology system (e.g., to one or more treatment delivery systems, treatment planning systems, imaging systems, and so on). In other examples, the query may be set to each element of the radiation oncology system in a stepwise order. For example, the query may first be sent to a first imaging system and, in response to receiving data from the first imaging system, the query may be sent to a second imaging system, and so on. The query may request information about a percentage and/or a numeric quantity of storage space of the queried element that is available to receive and store data from the computing device. The processing capability query includes a request for information about a capacity the queried element has to process received data. For example, this may include a request for information about a current processing load of the queried element, a number and/or size of data in a queue to be processed by the queried element, and/or an estimated time until the queried element is available to process the received data. The query further includes a request for a data format used by the queried element.

At 504, the method 500 includes receiving data from queried elements of the radiation oncology system. As described with respect to operation 502, the computing device may receive information about a processing capacity, a data format, and a storage capacity of each queried element from the respective queried element.

At 506, the method 500 includes defining and/or updating the model of the radiation oncology system. As described with respect to FIGS. 3-4, the model of the radiation oncology system includes each element (e.g., each imaging system, treatment delivery system, and treatment planning system) as an individual node, and relationships among elements are defined as edges among the nodes. The model further includes defined capabilities of each element (e.g., data transfer, processing, and/or storage capabilities). As the method 500 is continuously executed, the model may be updated to reflect changes in capabilities of each element and relationships therebetween. For example, an imaging system may be in use for a patient exam of a first patient and thus may not be available for data transfer for data of a second patient. In another example, an imaging system may be offline due to maintenance and/or repair, and is thus unavailable for data transmission. In a further example, a processing capability of an element may be full (e.g., the processor of the element does not have capacity for further processing). In another example, a storage space of an element may be full. The model may be updated to add and/or remove elements from the model, and/or to add and/or remove relationships among elements of the radiation oncology system.

At 508, the method 500 includes storing the model of the radiation oncology system. The model may be stored in a memory of the computing device. The method 500 may return to operation 502 to continuously send queries to elements of the radiation oncology system and use received data from the elements to update the model.

Turning to FIG. 6, a flow chart for the method 600 for sending information about a processing capacity, a data format, and an available storage space of a respective queried element to the computing device is shown. Method 600 may be executed according to instructions stored in memory of, and executed by one or more processors of, one or more elements of a radiation oncology system. For example, method 600 may be executed according to instructions stored in memory of and executed by one or more processors of the EMR database 150, the OIS 202, the CT system 210, the PACS 220, the LINAC system 226, and so on. The method 600 is described herein as being executed by the first imaging system 402 as an example. The method 600 may additionally or alternatively be independently executed by other elements of the radiation oncology system 200. For example, one or more steps of the method 600 may be executed by the first treatment delivery system 404 following execution of one or more steps of the method 600 by the first imaging system 402.

At 602, method 600 includes receiving a query from a computing device requesting information about an available storage space and a processing capability of an element of a radiology system, herein “the queried element”. In response to receiving the query, the queried element may identify and/or assign a metric value to one or more of the processing capability and the storage capacity of the queried element. The queried element may further define a data format used by the queried element.

At 604, method 600 includes sending information about the available storage space, the processing capability, and the data format from the queried element to the computing device. The information may be sent from the queried element via a wired and/or wireless connection. The method 600 ends, and may be repeated in response to the computing device sending another query to the queried element.

FIG. 7 shows a flow chart of the method 700 for a computing device that comprises consulting the model of the radiation oncology system and using the model to facilitate data transfer throughout the radiation oncology system. The model may be an example of a model generated according to the methods 300 and 500. The method 700 may be executed according to instructions stored in memory of, and executed by one or more processors of a computing device of the radiation oncology system. For example, the method 700 may be executed by the computing device 102.

At 702, the method 700 includes receiving a data output notice from an element of a radiation oncology system. The data output notice may be an indicator that the element of the radiation oncology system (e.g., an imaging system, a treatment planning system) has output data that is ready to be transferred to another element of the radiation oncology system. The data output notice may include information about a data format of the output data. In some examples, the data output notice includes the output data itself. The data output notice may further include indication of a desired process to be used to process the output data and/or one or more desired elements to transfer the output data to.

At 704, the method 700 includes selecting a data transfer path using the model of the radiation oncology system via which to transfer the output data to other elements of the radiation oncology system. The data transfer path may be selected for steps of a patient treatment workflow based on the relationships among elements of the radiation oncology system, processing capabilities, and available storage space of each element of the radiation oncology system. The patient treatment workflow includes ordered steps for patient treatment, and the data transfer path is selected to include elements of the radiation oncology system that are configured to execute steps of the patient treatment workflow efficiently. A patient treatment workflow may include different steps for different patients, and thus the data transfer path selected based on a particular patient treatment workflow of a patient may vary among patients. For example, a patient treatment workflow for a first patient includes: intaking patient information, performing a CT scan, storing CT scan data, and segmenting CT scan data. The data transfer path may start at a first element of the radiation oncology system that receives/intakes the patient information. The model of the radiation oncology system may identify the first imaging system 402 and the second imaging system 426 as being configured to perform a CT scan, as requested by the patient treatment workflow. Using the model of the radiation oncology system, the method 700 may select the first imaging system 402 or the second imaging system 426 to perform the CT scan, based on information about processing capabilities, data formats, and available storage space of each element.

At 706, the method 700 includes sending instructions to one or more element of the radiation oncology system to transfer data according to the selected data transfer path. For example, a first set of instructions may be sent from the computing device to the first element instructing the first element to send output data to a second element of the radiation oncology system (e.g., the second treatment planning system 420). In examples where a data format of the first element and the second element are different, the instructions may further direct the element that is sending data to transform the data to a data format of the element that is receiving the data. A second set of instructions may be sent from the computing device to the second element instructing the second element to transform and/or process data, and to send data to a third element of the radiation oncology system, as further described with respect to FIGS. 8 and 9. In further examples, a set of instructions sent to an element of the radiation oncology system may instruct the element to store received data.

FIG. 8 shows a flow chart of a method 800 for transferring output data according to transfer path instructions. The method 800 may be executed according to instructions stored in memory of, and executed by one or more processors of, one or more elements of a radiation oncology system. For example, the method 800 may be executed by the CT system 210, the PACS 220, the contouring module 222, and/or the treatment planning module 224 of FIG. 2. The element executing the method 800 may be selected based on which step of the patient treatment workflow the element is performing. For example, the method 800 may be performed by an element executing a first step of the patient treatment workflow that includes intaking and/or receiving patient information. The patient treatment workflow may include one or more additional steps following the first step. In this example, the method 800 may be performed by the treatment planning module 224 and includes sending data from the treatment planning module 224 to another element of the radiation oncology system to perform the second step of the patient treatment workflow, as further described herein.

At 802, the method 800 includes sending a data output notice to a computing device. As described with respect to FIG. 7, the data output notice that is sent by the first element executing the method 800 may include indication that data is ready to be output from the first element to another element of the radiation oncology system. The computing device may execute the method 700 in response to receiving the data output notice. The data output notice may further include information about a capacity of the first element, including a data format used by the first element.

At 804, the method 800 includes receiving transfer path instructions from the computing device. As described with respect to FIG. 7, the transfer path instructions may be a first set of instructions that instruct the first element to send output data to a second element of the radiation oncology system, and may further include instructions to transform a data format of the output data.

At 806, the method 800 optionally includes transforming the output data. The transfer path instructions received by the first element from the computing device may include an indication that a first data format used by the first element is different from a second data format used by the second element that the first element is to output the output data to. The computing device may instruct the first element to transform the output data from the first data format to the second data format prior to outputting the output data to the second element.

At 808, the method 800 includes outputting the output data. The first element may output the output data to the second element of the radiation oncology system as instructed by the transfer path instructions received from the computing device at operation 804. The method 800 returns to operation 802.

FIG. 9 shows a flow chart for the method 900 for an element of the radiation oncology system for transferring output data according to transfer path instructions. The method 900 may be executed according to instructions stored in memory of, and executed by one or more processors of one or more elements of a radiation oncology system. For example, the method 900 may be executed by the CT system 210, the PACS 220, the contouring module 222, and/or the treatment planning module 224 of FIG. 2. The element executing the method 900 may be selected based on which step of the patient treatment workflow the element is performing. For example, the method 900 may be performed by an element executing a second step of the patient treatment workflow that includes performing an imaging scan, and/or a third step of the patient treatment workflow that includes storing imaging data. In some examples, the method 900 may be performed by an element of the radiation oncology system that performs a last step of the patient treatment workflow. In this example, the method 900 may be performed by the PACS 220 and includes storing data, as further described herein. The method 900 may additionally be executed by a third element, a fourth element, and so on, as indicated in the data transfer path selected by the computing device. For example, the method 900 may be executed by the contouring module 222 and/or segmentation module of the radiation oncology system, and includes processing data according to the respective module and transferring and/or storing processed data as directed by steps of the patient treatment workflow.

At 902, the method 900 includes receiving transfer path instructions from the computing device. As described with respect to FIGS. 7-8, the transfer path instructions may be a second set of instructions that instruct the second element to send output data to a third element of the radiation oncology system, and may further include instructions to transform a data format of the output data. In further examples, the second set of instructions may instruct the second element to process the output data.

At 904, the method 900 includes receiving output data from the first element of the radiation oncology system. The first element and the second element may be communicably coupled via a wired and/or a wireless connection. For example, the first element and the second element may be physically positioned in the same room, in different rooms of the same healthcare environment, and/or in different healthcare environments.

At 906, the method 900 optionally includes processing the output data received from the first element. The second element may process the output data according to instructions for methods stored in a memory of and executed by a processor of the second element. For example, when the second element is a treatment planning system, processing the output data may include using one or more elements of the output data to generate a treatment plan.

At 908, the method 900 includes transferring and/or storing the output data according to the transfer path instructions. As described above, the transfer path instructions received from the computing device at operation 902 may direct the second element to send the output data (e.g., processed or not processed by the second element) to the third element of the radiation oncology system. The transfer path instructions may further instruct the second element to store the output data in a memory of the second element and/or to hold on transferring the output data until the second element receives further instructions. The method 900 returns to operation 902.

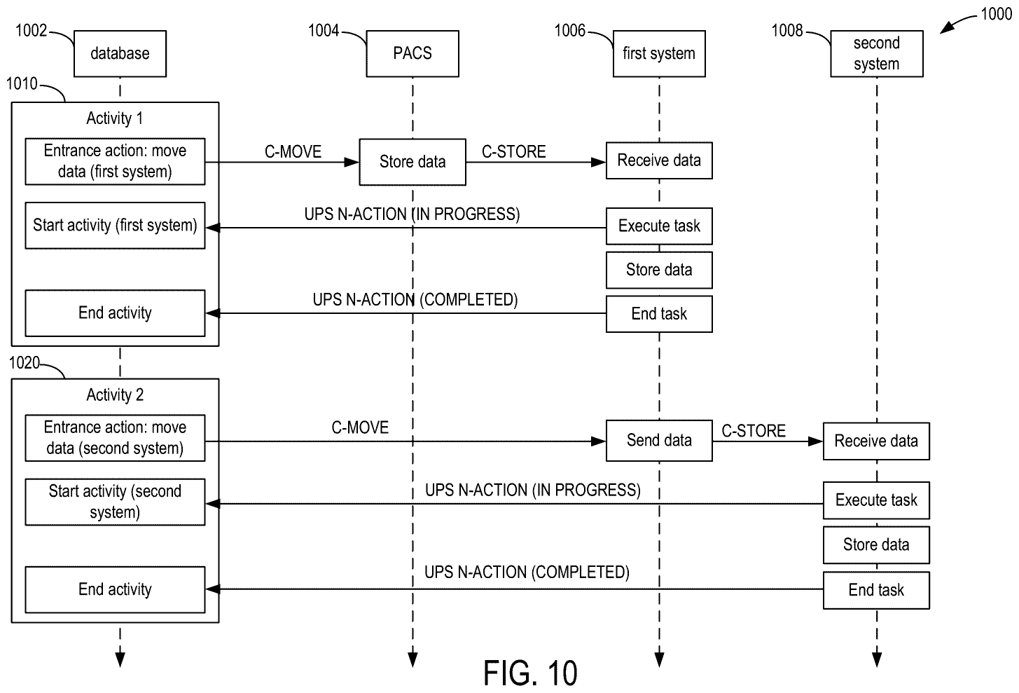

Turning to FIG. 10, an example timeline 1000 of data transfer according to the methods of FIGS. 3-9 is shown. The timeline 1000 shows elements of a radiation oncology system, including a computing device 1002 (e.g., the computing device 102 of FIGS. 1-2, 4), a PACS 1004 (e.g., the PACS 220 of FIG. 2), a first system 1006 (e.g., the CT system 210 of FIG. 2), and a second system 1008 (e.g., a segmentation module). For a first activity 1010, the computing device 1002 initiates an entrance action to identify a path for transferring data from the computing device 1002 according to a first step of a patient treatment workflow. The entrance action may include generating a model of the radiation oncology system and sending a C-MOVE query to each of the PACS 1004 and the first system 1006. The computing device 1002 may identify a path to the PACS 1004 as an efficient data transfer path for the patient treatment workflow, as described above with respect to FIGS. 3 and 4, and execute C-MOVE to transfer data to the PACS 1004, where the data is stored. As a further step of the identified data transfer path, data may be transferred to the first system 1006 by executing C-STORE. The first system 1006 may receive the data and execute a task, such as processing received data, and store processed data. The first system 1006 is in communication with the computing device 1002 while executing the task and indicates a status of the task via UPS N-ACTION. The first activity 1010 may thus automatically send data through the radiation oncology system along a transfer path according to the patient treatment workflow. The first system 1006 is selected to perform a process of the patient treatment workflow (e.g., perform a CT scan).

For a second activity 1020, the computing device 1002 initiates an entrance action to identify a path for transferring data from the computing device 1002 to an element of the radiation oncology system to execute further steps of the patient treatment workflow. The entrance action may include generating a model of the radiation oncology system and sending a C-MOVE query to the first system 1006 and the second system 1008. The computing device 1002 may identify a path to the first system 1006 as an efficient data transfer path, as described above with respect to FIGS. 3 and 4, and execute C-MOVE to transfer data to the first system 1006. However, as described above, capabilities of elements of the radiation oncology system may change and the model may be updated in real time. The computing device 1002 may transfer data from the first system 1006 to the second system 1008, by executing C-STORE, without using the first system 1006 to process the data. The second system 1008 may receive the data and execute a task, such as processing received data, and store processed data. The second system 1008 is in communication with the computing device 1002 while executing the task and indicates a status of the task via UPS N-ACTION.

The systems and methods described herein provide efficient transfer of data among elements of a radiation oncology system which may have different vendors, processing abilities, storage capacities, and/or data format standards. Automatic data transmission according to the model and a patient treatment workflow increases performance efficiency of one or more devices of the radiation oncology system. A processing demand and/or a storage demand placed on a device may be decreased by directing a request for processing and/or data storage to a different device that has a greater processing capacity (e.g., less processing power is being used for other operations) and/or a greater storage capacity. This may further increase processing efficiency by decreasing processing time and/or delays to processing and/or storage caused by imparting a processing and/or storage demand on a device that does not have the demanded processing capacity and/or storage capacity available. Using the adaptable model-based approach described herein allows data transfer paths to be adjusted in real-time as the model is updated based on changes to the radiation oncology system. In this way, a processing demand on each element of a radiation oncology system may be reduced, therefore data transfer time may be reduced compared to manual selection of elements used to process the data. The ability to transform data to formats which are compatible with different elements of the radiation oncology system allow for seamless transfer of information among systems, which may reduce a data transfer and processing time and increase data transfer accuracy. Data transfer among multiple elements may be accelerated and transfer of patient data for each patient may be tracked throughout the radiation oncology system.

The disclosure also provides support for a method for choosing a data transfer path, comprising: defining a model of a radiation oncology system, the model including elements of the radiation oncology system as nodes, and relationships therebetween as edges, storing the model in a non-transitory memory of a computing device, inducing a source workflow step to initiate a C-MOVE request by sending a query from a source system element to a destination system element to determine a processing capability and an available storage space of the destination system element, selecting a transfer path from the source system element to the destination system element based on the model of the radiation oncology system and the capability of the destination system element, transforming data into a format that is compatible with the destination system element, transferring transformed data to the destination system element according to the transfer path, updating the model of the radiation oncology system to reflect a real-time state of transfer, and storing an updated model in the non-transitory memory. In a first example of the method, selecting the transfer path includes comparing a processing power demand, number of steps, and transfer time among a two or more paths from the source system element to the destination system element as defined by the model, and selecting the transfer path at least one of a lowest processing power demand, a fewest number of steps, and/or a shortest transfer time. In a second example of the method, optionally including the first example, the method further comprises: comparing an efficiency of the transfer path to an updated model of the radiation oncology system and selecting a second transfer path from the source system element to the destination system element based on the updated model when the second transfer path is more efficient than the transfer path. In a third example of the method, optionally including one or both of the first and second examples, the method further comprises: transferring transformed data to the destination system element according to the second transfer path. In a fourth example of the method, optionally including one or more or each of the first through third examples, the format is DICOM®. In a fifth example of the method, optionally including one or more or each of the first through fourth examples, sending the query from the source system element to the destination system element comprises sending the query from an imaging system to a treatment planning system. In a sixth example of the method, optionally including one or more or each of the first through fifth examples, transformed data is transferred using at least one of a unified procedure setup (UPS-RS), a DICOM®-UPS, and/or a modality worklist adaptor. In a seventh example of the method, optionally including one or more or each of the first through sixth examples, storing the updated model comprises replacing the model in the non-transitory memory. In an eighth example of the method, optionally including one or more or each of the first through seventh examples, storing the updated model comprises updating the model in the non-transitory memory.

The disclosure also provides support for a radiation oncology system, comprising: a source system, a destination system, and a computing device having a processor and machine-readable instructions stored in non-transitory memory and executable by the processor to: define a model of the radiation oncology system, the model including the source system and the destination system as nodes, and relationships therebetween as edges, store the model in the non-transitory memory, induce a source workflow step to initiate a C-MOVE request by sending a query from the source system to the destination system to determine a processing capability and an available storage space of the destination system, selecting a transfer path from the source system to the destination system based on the model of the radiation oncology system and the capability of the destination system, transform data into a format that is compatible with the destination system, transfer transformed data to the destination system according to the transfer path, update the model of the radiation oncology system to reflect a real-time state of transfer, and store an updated model in the non-transitory memory. In a first example of the system, the source system is an imaging system. In a second example of the system, optionally including the first example, the destination system is a treatment planning system. In a third example of the system, optionally including one or both of the first and second examples, the capability of the destination system includes a data format of the destination system. In a fourth example of the system, optionally including one or more or each of the first through third examples, the data is medical images. In a fifth example of the system, optionally including one or more or each of the first through fourth examples, the destination system is a treatment delivery system.

As used herein, an element or step recited in the singular and preceded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method for choosing a data transfer path, comprising:
defining a model of a radiation oncology system, the model including elements of the radiation oncology system as nodes, and relationships therebetween as edges;
storing the model in a non-transitory memory of a computing device;
sending a query from a source system element to a destination system element to determine a processing capability and an available storage space of the destination system element;
selecting a transfer path from the source system element to the destination system element based on the model of the radiation oncology system and the processing capability of the destination system element;
transforming data into a format that is compatible with the destination system element;
transferring transformed data to the destination system element according to the transfer path;
updating the model of the radiation oncology system to reflect a real-time state of transfer;
and
storing an updated model in the non-transitory memory.

2. The method of claim 1, wherein selecting the transfer path includes comparing a processing power demand, number of steps, and transfer time among a two or more paths from the source system element to the destination system element as defined by the model, and selecting the transfer path at least one of a lowest processing power demand, a fewest number of steps, and/or a shortest transfer time.

3. The method of claim 1, further comprising comparing an efficiency of the transfer path to the updated model of the radiation oncology system and selecting a second transfer path from the source system element to the destination system element based on the updated model when the second transfer path is more efficient than the transfer path.

4. The method of claim 3, further comprising transferring transformed data to the destination system element according to the second transfer path.

5. The method of claim 1, wherein the format is digital imaging and communications in medicine (DICOM®).

6. The method of claim 1, wherein sending the query from the source system element to the destination system element comprises sending the query from an imaging system to a treatment planning system.

7. The method of claim 1, wherein transformed data is transferred using at least one of a unified procedure setup (UPS-RS), a DICOM®-UPS, and/or a modality worklist adaptor.

8. The method of claim 1, wherein storing the updated model comprises replacing the model in the non-transitory memory.

9. The method of claim 1, wherein storing the updated model comprises updating the model in the non-transitory memory.

10. A radiation oncology system, comprising:
a source system;
a destination system; and
a computing device having a processor and machine-readable instructions stored in non-transitory memory and executable by the processor to:
define a model of the radiation oncology system, the model including the source system and the destination system as nodes, and relationships therebetween as edges;
store the model in the non-transitory memory;
send a query from the source system to the destination system to determine a capability and an available storage space of the destination system;
selecting a transfer path from the source system to the destination system based on the model of the radiation oncology system and the capability of the destination system;
transform data into a format that is compatible with the destination system;
transfer transformed data to the destination system according to the transfer path;
update the model of the radiation oncology system to reflect a real-time state of transfer; and
store an updated model in the non-transitory memory.

11. The radiation oncology system of claim 10, wherein the source system is an imaging system.

12. The radiation oncology system of claim 10, wherein the destination system is a treatment planning system.

13. The radiation oncology system of claim 10, wherein the capability of the destination system includes a data format of the destination system.

14. The radiation oncology system of claim 10, wherein the data is medical images.

15. The radiation oncology system of claim 10, wherein the destination system is a treatment delivery system.

16. A method, comprising:
receiving a patient treatment workflow including an ordered series of steps directed to monitor a patient, provide treatment to the patient, and/or plan treatment for the patient;
defining a model of a radiation oncology system;
identifying one or more elements of the radiation oncology system configured to perform each step of the ordered series of steps of the patient treatment workflow;
selecting a data transfer path through elements of the radiation oncology system using the model of the radiation oncology system, where the data transfer path includes an ordered series of data transmissions among elements of the radiation oncology system that correspond to the ordered series of steps of the patient treatment workflow; and
automatically directing data transmission among elements of the radiation oncology system, according to the ordered series of steps of the patient treatment workflow and the data transfer path.

17. The method of claim 16, wherein defining the model of the radiation oncology system includes identifying communication connections among elements of the radiation oncology system.

18. The method of claim 16, wherein identifying one or more elements of the radiation oncology system configured to perform each step of the ordered series of steps of the patient treatment workflow includes querying a processing capability, a data format, and a storage capability of each element.

19. The method of claim 16, wherein selecting the data transfer path includes comparing a processing capability, a data format, and a storage capability of one or more elements of the radiation oncology system that are configured to execute a same step of the patient treatment workflow, and selecting an element of the one or more elements that has a desired processing capability, data format, and/or storage capability.

20. The method of claim 19, wherein a selected element of the one or more elements with has a relatively highest processing capability and/or storage capability of the one or more elements.
